(19) United States
(12) Patent Application Publication
(10) Pub. No.: US 2003/0235581 A1
(43) Pub. Date: Dec. 25, 2003

(54) Ligands for Reproductive Science

(76) Inventor: George Pieczenik, New York, NY (US)

Correspondence Address: DR. GEORGE PIECZENIK, APT. 1F, 412 EAST 55TH STREET, NEW YORK, NY, 10022, US

(21) Appl. No.: 10/604,016

(22) Filed: Jun. 20, 2003

Related U.S. Application Data
(60) Provisional application No. 60/319,332, filed on Jun. 20, 2002.

(51) Int. Cl. A61K 39/395

(52) U.S. Cl. 424/130.1

(57) ABSTRACT

Identification, Isolation and Use of Specific Ligands for Reproductive Science

### CROSS REFERENCE TO RELATED APPLICATIONS

Priority is claimed under 35 USC 119 to Provisional Patent No. 60319332 “Ligands for Reproductive Science” filed Jun. 20, 2002 for conception and for constructive reduction to practice and additionally for conception priority is claimed to USPTO Disclosure Document-511258 “Ligands to Identify Viable Human Oocytes and Embryos” filed on May 14, 2002.

### FEDERAL RESEARCH STATEMENT

[No federal research funds were used in this research]

### BACKGROUND OF INVENTION

Classical pharmacology operates from the outside inward, that is drugs are targeted to surface receptors on cells. This requires isolating the receptor or the coding (gene) for the receptor. This invention creates a paradigm shift based on the new knowledge that the human genome has a limited number of coding sequences. The relationship between these coding sequences and final gene protein product is still to be uncovered. This invention will assist in the elucidation of this unknown area, but in principle, products can be obtained without actually understanding the exact relationship between coding and protein.

Cell surface changes reflect physiological status. This invention allows ligands that interact with surface markers to be developed without actually having to directly isolate surface receptors. This invention allows for a new form of pharmacology which operates from the inside outward. Initially this invention will be applied to human gametes and embryos for developing diagnostic tools without extracting cells. These will be aimed at predicting viability and general health. This invention will lead to new avenues of therapy and proprietary products. Simply put, this invention allows us to create a set of viability stains derived solely from genomic information without access to any actual substrate. These viability stains are in turn lead compounds for therapeutic compounds.

### SUMMARY OF INVENTION

1) A proteome lignd library of the genome: The human genome has a limited number of coding sequences, in the range of 19,500 28,000. It is possible to chemically synthesize parts of proteins corresponding to these sequences without knowing the actual complete protein composition or function. Each one of these peptide sequences can be exposed to a varied peptide ensembles in the range of 10³ to 10⁹ combinations of smaller sequences. These ensembles are called a combinatorial library. These libraries can be made on the surface of phage, bacteria, or chemically synthesized. These combinatorial libraries can be both small peptide libraries or antibody fragment libraries. These libraries were first invented by one of the inventors on this patent and has issued as U.S. Pat. No. 5,866,363 and its continuation (Pieczenik, Feb 2, 1999) which are hereby incorporated by reference.

This invention employs, inter alia, these combinatorial libraries to bind genomic “targeted” sequences and develop “targeting” ligands for each of the synthetic genomic targets. A synthetic genomic target is hereby defined as a synthesized peptide sequence based on the known nucleotide sequence of a identified coding region with or without known function. The size of the possible universe of such synthetic genomic targets can be as large as the genome itself where every part of every coding sequence is a target or just a representative universe of the genome where 3 subsections of each coding is a synthetic genomic target. For example, as there are in the range of 20,000 30,000 codings and we can take 3-5 sample targets per coding, this invention allows us to create 60,000 to 150,000 differential targeting ligands for the human genome and other genomes. This set of differential targeting ligands represents the targeting universe for the humane and other proteome. These differential targeting ligands are also lead compounds for pharmaceuticals. These differential targeting ligands are also informational ligands to allow homology searches of the genome to identify naturally occurring homologous sequences in order to identify the naturally occurring corresponding ligand.

This mode of this invention is to generate a representative ligand binding set for the whole genome.

2) Ligands to identify viable human gametes, embryos and stem cells, i.e. non-toxic viability stains. Another mode of this invention is to generate a representative ligand binding set related to embryological and reproductive functilibaivtions.

Another mode of this invention is reversible and non-reversible binding ligands.

Another mode of this invention are such ligands which can be used to characterize oocyte and embryo viability and quality. As such, these ligands may be related to apoptotic and anti apoptotic functions.

Another mode of this invention is to identify those oocytes and embryos that are most likely to form a successful and healthy pregnancy.

The utility of this invention is that one can assess the genetic status and/or developmental viability of embryos without the need for embryo biopsy.

Another mode and utility of this invention is the non toxic characterization and identification of human gametes (sperm and oocytes).

Another mode and utility of this invention is the non toxic characterization and identification of stem cells.

An enablement mode of this invention, which can also allow the correlation of structure to function, is to synthesize genomic codings which are characteristic of oocyte and embryo viability. Certain genomic sequences and, also, genomic products which bind such DNA sequences, are characteristic of viable embryos. All these targets either protein or nucleic acid which are related to embryological and reproductive functions are to called “reproductive target ligands”. Those ligands designed either from the literature, calculation or from binding with combinatorial libraries which bind “reproductive target ligands” are to be called “reproductive targeting ligands” Some representative “reproductive target ligands” which have been identified are described at the end of this section.

Some representative “reproductive targeting ligands” which have been identified are described at the end of this section.

Some pairs of “reproductive target ligands” and their corresponding “reproductive targeting ligands” which have been identified and isolated are described at the end of this section also.

The peptide sequences or specifically the “reproductive targeting ligands” identified with either recombinant or chemical combinatorial libraries as possible binding ligands can be made either recombinantly or chemically. These ligands can be labeled appropriately with FITC, XITC, rhodamine or other stains for visual localization and other labels for other types of detection, such as, NMR (using gadolinium, inter alia), electron microscopy (heavy metal i.e. gold, mercury, inter alia), echo, sonar, etc. inter alia.

Potential sequences which can be informatively deduced to have some relationship to embryo implantation and viability can be synthesized fully or in part and become a “reproductive target sequence” and have their “reproductive targeting ligands” made as described above.

One can estimate that there may be in their range of anywhere from 1-200 such implantation markers and the corresponding ligands may be in the range of 3-600. This invention allows for the design of kits for the detection of these ligands and the use of these ligands in determining implantation characteristics. These ligands are vital stains and are easily removable. Said kits will allow embryo viability detection during assisted reproductive treatments.

In addition, because of the constrained nature of molecular evolution, the types of genes that are involved in embryo implantation and development will necessarily overlap with classes of genes that are involved in tumor genesis. It is therefore inevitable that ligands identified by this invention for embryological and reproductive markers will find application in cancer research and diagnosis. Classically, such a relationship has already been defined for the carcino-embryonic antigen, CEA, which is used for prostate cancer detection.

### BRIEF DESCRIPTION OF DRAWINGS

FIG. 1. Target Ligand Calculated from Combinatorial Library Constraints Sperm Head Ligand

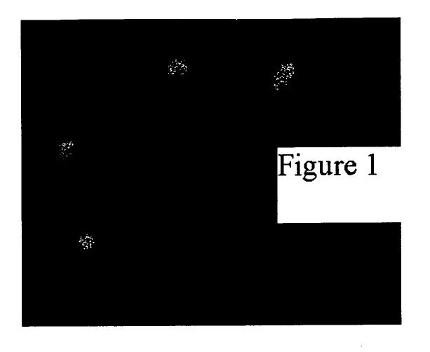

FIG. 2. Sperm Head Ligand as Target Ligand Egg Ligand

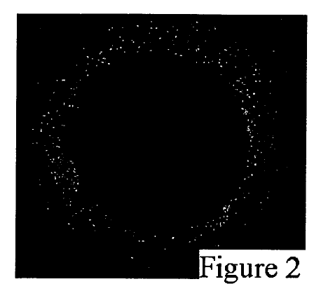

FIG. 3. Sperm Neck Ligand from Direct Binding to Combinatorial Library

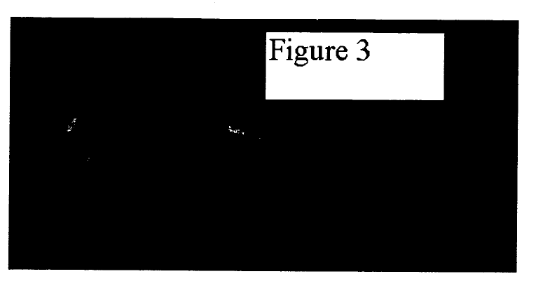

### DETAILED DESCRIPTION

Initial targets: The following “reproductive target ligands” are identified because their functionand structure are known to be related in some fashion to reproduction, embryological development and or tumorigenisis. They are BRAC1, BRAC2, PED (HLA-E, HLA-F, HLA-G), Mad1, Mad2, Oct4, Bub1, Bub2, ATM, P53, HLA-C, HLA-1, REC8 (cohesin), hMLH1, PMS2, SPINDLIN, NOS1-3, IGF2, CSF-1/c-fms, SF/c-kit, STK15, APC, RB1, SC1, Survivin, INCENP, Auroral, Dnmt1o, SAMP32, YLP12, TSA-1, Sptrx, Sptrx-2, GDF-9, CDC25C, HSP, hTCS, leptin, STAT 3, Cadherin, Catenin, Cyclin, Integrin RGD-receptors, Non RGD-Integrin receptors, apoptotic pathway proteins Bcl-2 i.e. Bax.

These genes have various functions of significance for gametogenesis, fertilization or the development of the human preimplantation embryo. Cellular mechanisms governed by these genes include: cell cycle regulation; maintenance of DNA or chromosomal integrity; meiotic recombination; embryo polarity; apoptosis; control of gene expression (including imprinting); sperm-egg interactions. The DNA and protein sequences of the genes listed above are already characterized, and using this invention allows us to design binding ligands to stain, identify. stimulate or block the proteins produced.

Our gene expression data confirms that preimplantation development is accompanied by temporal fluctuations in the activity of specific genes. The differences in expression observed reveal that certain genes are activated or switched off at precise moments and indicate that they may be co-coordinating specific aspects of development. Furthermore, it has been found that abnormal embryo morphology and impaired viability is correlated with aberrant expression of specific genes. This invention allows for the creation of ligands that bind the proteins produced by developmental genes provides a powerful class of research tools for studying the proteins insitu. Said ligands can be made available as a kit. The utility of said kits is that they can serve as vital stains, thereby, potentially providing a means to assess the expression of critical genes in living gametes or embryos. The additional utility is that said kits allow the design of simple clinical tests aimed at assisting embryologists in the identification of the embryos most likely to form a successful pregnancy, as well as revealing the biological pathways important during gametogenesis, fertilization and embryo development.

3) DNA probes and Nucleic Acid Sequences as “Target-Ligands” The chromosome detection techniques currently applied to embryos utilize double stranded DNA probes in the process known as fluorescence in situ hybridization (FISH). Probes usually exceed 60 Kb, are directly or indirectly labeled with fluorochromes and hybridize to specific regions chromosomes. This allows the enumeration of chromosomes contained within interphase nuclei, revealing problems such as aneuploidy. This process is extensively patented, specifically, the use of blocking DNA. FISH is used extensively for both prenatal and preimplantational genetic diagnosis, cancer genetics and many other areas of research.

As an alternative to DNA and PNA probes, this invention allows for the creation of “targeting peptides” capable of binding to specific chromosomal regions could be used for chromosome enumeration. This may be possible as unique DNA sequences extend over large areas of individual chromosomes affecting the chromatin structure and protein binding characteristics of the DNA. In addition, the relationship between chromosomal regions that are coding and the consequent localization of the peptides coded by these regions on the surface of the embryo will be deciphered by these ligands. Peptide ligands directed to chromatin structure rather than sequence information can also be developed. In this case, the target will not be a peptide sequence but a DNA sequence or structure.

4) Gamete and embryo screening: This invention allows for “reproductive targeting ligands” to compare, contrast and screen gametes and embryos from various sources. This invention allows ligand binding patterns to be evaluated in terms of embryo morphology, chromosomal integrity and development rate.

To show that the inventors have complete conceptual possession of this invention and its potential, this invention allows for developing the following diagnostic products related to sperm: ligands for identifying chromosomally normal sperm, ligands for sperm sexing, ligands for acrosome reacted sperm, ligands for sperm identification, ligands for centrial competence. This invention allows for developing the following diagnostic products related to oocyte: ligands for identifying chromosomally normal oocytes, ligands for identifying cytoplasmic maturity, ligands for identifying fertilizing and implanting potential, ligands for identifying zona integrity and ligands for identifying cryosurvival. This invention allows for developing the following diagnostic products related to embryos: ligands for identifying chromosomally normal embryos, ligands for identifying cytoplasmic maturity, ligands for identifying the cortical reaction, ligands for identifying implanting potential, ligands for identifying zona hardening and ligands for identifying cryosurvival. This invention allows for developing the following therapeutic products: for sperm-identifying lead compounds for sperm contraception and identifying and blocking HIV infected sperm; for oocyte—developing maturational peptides and peptide-mimetics and ligands for identifying the cortical reaction; for embryos—identifying surface markers for genetic disease, agonist/antagonist for embryo viability and development and serendipidous findings related to oncological products.

We claim:

1. A kit of sperm diagnostic products comprising ligands for identifying chromosomally normal sperm, ligands for sperm sexing, ligands for acrosome reacted sperm, ligands for sperm identification and ligands for centriol competence and combinations thereof.

2. A kit of oocyte diagnostic products comprising ligands for identifying chromosomally normal oocytes, ligands for identifying cytoplasmic maturity, ligands for identifying fertilizing and implanting potential, ligands for identifying zona integrity and ligands for identifying cryosurvival.

3. A kit of embryo diagnostic products comprising ligands for identifying chromosomally normal embryos, ligands for identifying cytoplasmic maturity, ligands for identifying the cortical reaction, ligands for identifying implanting potential, ligands for identifying zona hardening and ligands for identifying cryosurvival.

4. A sperm therapeutic method comprising the step of identifying lead compounds for sperm contraception. A sperm therapeutic method comprising the steps of identifying HIV blocking peptides and then using said peptides to block the infection of sperm.

5. An oocyte therapeutic method comprising the steps of identifying and developing maturational peptides and peptide mimetics.

6. An oocyte therapeutic method comprising the step of identifying ligands for the cortical reaction.

7. A method for genetic disease identification comprising the steps of identifying, isolating and applying embryonic surface markers for genetic disease.

8. A product comprising antagonistic ligands for embryo viability and development.

9. A product comprising agonistic ligands for embryo viability and development.

10. A reproductive targeting ligand comprising an Oct 4 binding ligand, said Oct 4 binding ligand having the sequence RIM, inter alia.

11. A reproductive targeting ligand comprising a sperm head ligand, said sperm head ligand having, inter alia, the amino acid sequence HEHR.

12. A reproductive targeting ligand comprising a oocyte binding ligand, said oocyte binding ligand having, inter alia, the amino acid sequence HNSS.

13. A binding pair of reproductive targeting and target ligands comprising the sperm head ligand of claim 11 and the oocyte binding ligand of claim 12 respectively.

14. A reproductive targeting ligand comprising a bax binding ligand, said bax binding ligand having, inter alia, the sequence VPMLK.

15. An embrylogical ligand with oncological properties.
